(19) United States
(12) Patent Application Publication
(10) Pub. No.: US 2004/0152195 A1
(43) Pub. Date: Aug. 5, 2004

(54) Vector for introducing molecules in eukaryotic cells and molecules vectorised by same

(76) Inventor: Denis Michel, Rennes (FR)

Correspondence Address: Robert M Angus, Westman Champlin & Kelly, International Center Suite 1600, 900 Second Avenue South, Minneapolis, MN, 55402-3319, US

(21) Appl. No.: 10/466,591

(22) PCT Filed: Jan. 18, 2002
(86) PCT No.: PCT/FR02/00217

(30) Foreign Application Priority Data
Jan. 19, 2001 (FR) 01/00773

(51) Int. Cl. C12N 15/85

(52) U.S. Cl. 435/455; 435/320.1

(57) ABSTRACT

The invention concerns a vector for introducing a molecule (X) having at least an amino-terminal end into a eukaryotic cell, characterised in that it consists of an association of a cell incorporating module enabling membrane translocation, and an uniquitin module or other related peptide capable of conjugation, said vector having a carboxy-terminal end designed to be fused by peptide bond to the amino-terminal end of said molecule (X). The invention is characterised in that said vector is rapidly destroyed after separation from said molecule (X). The invention also concerns any molecule vectorised by such a vector.

The present invention relates to the field of biochemistry, cellular biology and medicine.

More precisely, the invention relates to a tool molecular, hereinbelow referred to as “vector”, to be used for introducing any type of molecule to eukaryotic cells.

The invention finds application in particular for intracellular incorporation of amino acids, or peptides of any size and sequence (di-peptides, tri-peptides, polypeptides, proteins . . . ) and/or capable of being modified chemically.

The invention also relates to any molecule vectorised by such a vector. It is noted that in the present description the term “vectorised molecule” is understood to mean the whole constituted by the vector fused with the molecule to be transported into the cell.

In accordance with the prior art, the contribution of molecules to the eukaryotic cells can be made by fusing the molecule to be transported with cell-incorporating incorporating modules (cell-permeable modules” CPM), such as membrane translocation polypeptides, capable of crossing the cell membranes and gaining direct access to the cytoplasmic cell compartment.

Several CPMs are already known and used for introducing peptides and proteins which do not spontaneously cross over the membranes of the eukaryotic cells. The CPM can correspond to domains of natural proteins (Schwarze and coll. 2000. Trends Cell Biol. 10, 290-295, Fujihara and Nadler 1999. EMBO J. 18, 411-419) or else to peptides of artificial sequences (Futaki and coll. 2000. J. Biol. Chem. 276, 5836-5840; Ho and coll. 2001. Cancer Res. 61, 474-477).

Several properties of translocation peptides described according to the prior art make promising tools of them in cellular and medical biology, such as for example:

an efficacious passage through the hemato-encephalic barrier (tight junctions), at the same time retaining its integrity.

the capacity to distort the resistance system to drugs (MDR), by which tumour cells become resistant to many anticancer agents.

direct passage into the cytoplasmic compartment, independent of endocytosis phenomena. This particularity, sparsely distributed among other modes of non-viral vectorisation, enables efficacious cellular incorporation at low temperature, and avoids the problem of endosomes moving out towards the cytoplasm.

rapid vectorisation in all the regions of the organism a short time after intravenous injection, preventing prolonged presence in the blood.

possible coupling of these peptides to non-peptidic compounds and to nucleic acids, which are hydrophilic and do not spontaneously cross cell membranes.

The main application of the prior art is the introduction of proteins or peptides into cells, by means of CPM constituted by translocation peptides. According to the prior art, proteins or peptides (X) to be introduced to the cells are prepared in fusion with CPM via a peptide bond, in the form of recombinant CPM-X proteins. These proteins are produced in appropriate expression systems, for example in bacteria, or else synthesised by means of peptide synthesisers. When they are placed in contact with cells, CPM-X recombinant proteins are integrated into the cells, and the X part can exert a biological activity for which it is designed (Schwarze and coll. 2000. Trends Cell Biol. 10, 290-295).

The simple CPM-X fusions have several disadvantages:

the fact that the CPM remains associated with the X molecule after cellular incorporation can prevent good intracellular functioning of the transported (X) molecule, by diminishing the specificity and affinity of interaction of X with its intracellular targets, and by disfavouring its authentic three-dimensional folding. These negative interferences are particularly probable in the event where X is of a small size, such as a peptide.

the fact that the CPM remains associated with the X molecule after cellular incorporation can also prevent appropriate subcellular localisation of X. For example, many currently known translocation peptides have the tendency to accumulate in certain subcellular compartments (nuclear localisation associated with “NLS” activity, secretion etc.), bringing the X molecule with them. Inversely, fusion with a vector can prevent functioning of certain localisation sequences which would have been prevented in the transported X molecule, such as for example a mitochondrial N-end addressing signal.

the CPM can have, beyond a certain concentration, a certain degree of toxicity for the cells and organism, so as to limit its usage in cellular and medical biology. The possible toxicity can originate from destabilising properties of membranes of certain translocation peptides, such as for example fragments of proteins of viral envelopes or bacterial toxins, or else the presence of electrically charged fields such as for example translocation peptides rich in basic amino acids, or again poly-histidine sequences, labelling and purification by affinity for metals. Finally, the different modules of the vectorised molecule other than the transported X molecule, can have immunogenicity risks.

Such limits of the prior art constitute the disadvantages.

The object of the present invention is to propose a technique for introducing molecules into eukaryotic cells utilising CPMs but having the advantages novel relative to the prior art.

In particular, an object of the present invention is to propose a vector enabling any type of molecule and especially any type of peptide to be directed into eukaryotic cells, including very short peptides such as simple amino acids, dipeptides or tripeptides, but also peptides whereof the first amino acid is not methionine.

These objects are accomplished due to the invention which concerns a vector for introducing into a eukaryotic cell a (X) molecule having at least an amino-terminal end characterised in that it is constituted by association of a cellular incorporation module enabling membrane translocation, and a ubiquitin module or other related peptide capable of conjugation, said vector having a carboxy-terminal end for fusing by peptide bond to the amino-terminal end of said (X) molecule.

It is noted that in present description the terms “ubiquitin or other related peptides capable of conjugation”, hereinbelow called “UBL”, are understood to designate ubiquitin or related peptides such as NEDD-8, SUMO (Small Ubiquitinrelated MOdifier), UCRP (Ubiquitin Cross-Reacting Protein), RUB (Related to UBiquitin) and others (Jentsch and Pyrowolakis 2000. Trends Cell Biol. 10,335-342). The use of modified or shortened versions of UBL is possible if their capacity to introduce cleavage at their C-terminal end is retained.

Preferably, UBL is ubiquitin.

The UBL are naturally synthesised by fusing with carboxy-terminal extensions, constituted by simple amino acids, polypeptides or proteins. There are cellular enzymatic activities capable of precisely cleaving between the UBL fields and their C-terminal extensions.

In the case of ubiquitin the existence of such activities has been proven for a long time (Lund and coll. 1985. J. Biol. Chem. 260, 7609-7613; Pickart and Rose 1985. J. Biol. Chem. 260, 7903-7910).

Responsible enzymes have been identified (Mayer and coll. 1989. Biochemistry 28, 166-172). However, knowledge and possession of the enzymes responsible for these cleavage activities are not at all necessary to implement the present invention.

Several usages of these naturally activities have already been described in the domain of biotechnology. The present invention benefits from the natural existence of these enzymes in cells for a novel and original object: intracellular cleavage of pre-synthesised molecules inside the cells, after incorporation by the cells.

Within the scope of the present invention, the presence of a UBL module on the N-end part of the transported X molecule effectively enables cleavage between UBL and X, and thus release of the X molecule from the rest of the vector.

Utilisation of C-terminal cleavage of the UBL to attain this object has several advantages:

the natural activities responsible for this cleavage are spread over the majority of the eukaryotic cells and are constitutively active, allowing usage of this vector system to be generalised.

C-terminal cleavage of the UBL is conditioned only by recognition of the sequence in amino acids of the UBL module, situated exclusively on the NH₂ side of the cleavage site, enabling introduction of any sequence on the COOH side. This property ensures that as to the nature of the X peptides no condition or restriction is imposed. In particular, very short peptides, such as dipeptides, can also be effectively transported according to the present invention.

once cleavage has taken place precisely on the C-terminal end of the UBL, there is no constraint as to the nature of the N-end amino acid of the X molecule. In particular, this amino acid may be different to methionine, which is not the case for peptides synthesised from transgenes, following transcription and intracellular translation.

Utilisation of modified or abbreviated versions of UBL is possible if their capacity to induce cleavage at their C-terminal end is retained.

A very large number of types of (X) molecules can be vectorised by the vector according to the present invention and particularly simple amino acids and peptides of any size, and independently of any constraint in composition and sequence. The vector according to the present invention also enables chemically modified peptides to be vectorised.

The vector according to the invention can also be used for cellular incorporation of non-peptidic molecules, such as for example oligonucleotides or hydrophilic chemical compounds which will have been previously grafted to the carboxy-terminal end of an oligopeptide.

It is also seen that it will be possible to chemically modify the vectorised molecules, providing the peptide bond between the vectorised molecule and the vector remains accessible to UBL C-terminal hydrolases. By way of example, C-terminal acylation of the (X) molecule will allow its intracellular membrane anchorage.

The vector according to the present invention also has advantages relative to the peptides simply associated with membrane translocation peptides such as mentioned hereinabove.

More precisely, the present invention enables intracellular release of transported (X) molecules, in a form totally free of any fusion after cleavage between UBL and X.

The physical dissociation between the X molecule and the rest of the vector according to the present invention is of interest on several counts:

a— absence of functional interference between the vector and the X molecule.

If the X molecule has to assure biological function in the cells, the efficacy of this function can be considerably lessened if the other modules of the vectorised molecule remain associated (CPM, UBL and other). By steric encumbrance these modules can impede:

specificity and affinity of interaction of the X molecule with endogenous molecules of the cell, such as therapeutic targets. This latter point is particularly probable in the event where X is a peptide.

authentic three-dimensional folding of the X molecule.

appropriate subcellular localisation of the X molecule. Many currently known translocation peptides have the tendency to align with certain localisations (nuclear localisation by NLS activity (Nuclear Localization Signal'), resecretion etc.), bringing with them the transported X molecules. Inversely, fusion with a vector can prevent functioning of certain localisation sequences which would have been provided in the X molecule, such as for example a mitochondrial N-end addressing signal.

b— In the event where the X molecule is an peptide, this system permits total freedom of choice over the nature of this peptide. The intracellular release mechanism induced by the UBL module enables composition and sequence of any size to be delivered to the peptides cells, in particular those having desired amino- and carboxy-terminal regions, including having an amino acid amino-terminal other than methionine. By way of comparison it should be recalled that peptides synthesised in situ from transgenes are obligatorily synthesised with a starter methionine in an amino-terminal position, and that the transgenes present poorly to synthesis of small proteins and peptides.

c— Finally, intracellular dissociation between the X molecule and the modules of the vector enable introduction of an additional device into this vectorisation system: the ulterior destruction of all the modules of the vectorised molecule other than the transported X molecule (CPM, UBL and others). The programmed destruction of these modules constitutes the second objective of the present invention. In the simplest case where the whole of the compounds of the vector is peptidic in nature, this objective is attained by introduction of a module into the vectorised molecule.

Several categories of destabilisation modules can be used for this system, especially destruction boxes present in certain naturally unstable proteins such as for example cycines (Glotzer and coll. 1991. Nature 349, 132-138), or else an amino-terminal end exposing a destabilising amino acid followed by a region comprising one or more lysines.

Preferably, it is this latter type of destabilisation module which is used. The destabilising N-end module (DN, Destabilising N-end) is situated on the amino-terminal end of the vectorised molecule.

The nature of this type of N-end destabilising module rests on the determination that the half-life of intracellular proteins is very precisely determined by the nature of the N-ends sequences and principally by the nature of the first N-end amino acid (Bachmair and coll. 1986. Science 234, 179-186). We propose exploiting this natural activity of the eukaryotic cells in a novel and original manner, to program the intracellular destruction of a protein pre-synthesised outside the cells, then incorporated by the cells.

According to the DN selected, it is possible to program the destruction, following release of the transported X molecule, of the rest of the recombinant protein (comprising at least DNCPM-UBL).

It is important to note that the destabilising DN signals have an effect only in the intracellular context, and thus do not in any way affect the stability of the recombinant protein in the extracellular fluids. This point is demonstrated by the existence of numerous extracellular proteins which are naturally very stable, although having a destabilising amino acid at their amino-terminal end, exposed after cleavage of the peptide secretion signal.

We propose several methods to expose destabilising amino acids at the N-end of the vectorised protein:

A— By enzymatic cleavage of the recombinant protein by a protease:

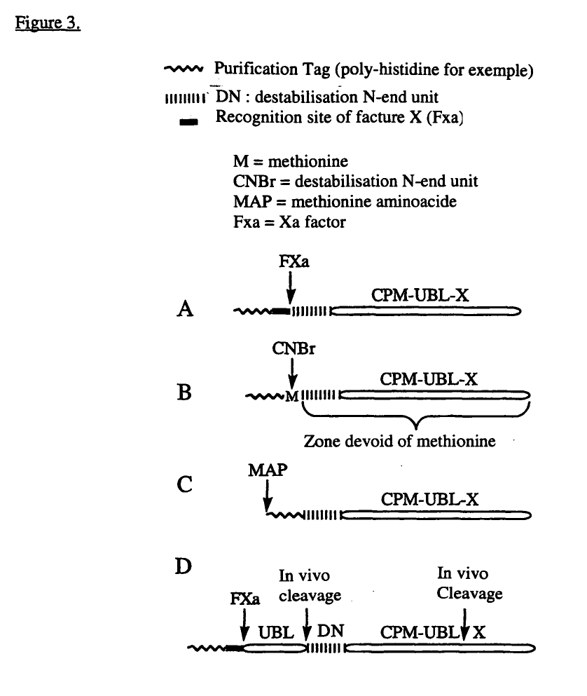

or a protease whereof the recognition site is localised exclusively from NH₂ of the cleavage site (such as for example the Xa factor), having provided a destabilising residue just downstream of its cutting site (FIG. 3A).

or a protease cutting inside its recognition unit, though selected because the amino acid forming part of its recognition site and situated just downstream of its cutting site, happen to be a destabilising residue.

B— By chemical cleavage, for example with cyanogen bromide (CNBr), at the level of a methionine of the purified recombinant protein, having provided a destabilising amino acid just downstream in the vectorised molecule. A prior condition to this method is where the recombinant protein is conceived such that it contains no other methionines (FIG. 3B).

C— By enzymatic elimination of the initiating methionine N-end of the purified recombinant protein, by an appropriate methionine amino peptidase (MAP) (FIG. 3C).

D— Alternatively, a destabilising N-end amino acid can be exposed not during preparation of the recombinant protein as previously, but by the receptor cells themselves. For this, an additional UBL module is inserted into the recombinant protein just upstream of the DN (FIG. 3D). This method is complex but does offer the advantage of guaranteeing that destruction of the vector module will not take place until after C-terminal cleavage of the UBL, and thus after release of the transported X molecule. In this case, it is possible to select a destabilising amino acid dictating a very short protein half-life (such as for example arginine).

Methods A, B and D are preferably used. In the case of method B, it is appropriate to first avoid the presence of internal methionines in the recombinant protein. In particular, methionine corresponding to the first UBL amino acid is replaced by leucine.

It is assured that degradation of the vector module will be quite different relative to the release of the transported molecule, in two ways;

a— by selecting a DN and in particular an amino acid N-end, causing destabilisation over a period significantly greater than the action period for UBL C-end hydrolases.

b— by the strategy of exposing DN by cleavage of UBL placed upstream according to point D of the methods for inserting a DN, as illustrated in FIG. 3D. In this method the additional UBL module is not destroyed in the cells, but is totally inoffensive where it is identical to normal endogenous cellular UBLs.

Several molecules likely to be used within the scope of the present invention to constitute the UBL module are known, such as ubiquitin, SUMO, NEDD-8, UCRP, RUB or others (see Jentsch and Pyrowolakis, 2000. Trends Coll. Biol. 10, 335-342). As already mentioned hereinabove utilising modified or shortened versions of UBL within the scope of the present invention is also possible if their capacity to induce cleavage at their C-terminal end is retained.

Preferably this module is ubiquitin.

Several methods likely to be used to constitute the destabilising intracellular module of the vector are known.

Preferably this module is made up of a destabilising amino acid terminal followed by a region comprising one or more lysine residues. The destabilising amino acid amino terminal is preferably glutamine.

It is noted also that within the scope of the present invention said incorporation module is preferably a membrane translocation peptide such as a peptide derived from the TAT protein of the AIDS virus (HIV), of a natural or modified sequence.

The invention also relates to any vectorised molecule, characterised in that it is constituted by fusion of a (X) molecule having at least one amino—terminal end whereof said amino-terminal end is fused by peptide bond to the carboxy-terminal end of the vector such as described hereinabove.

According to a variant, said (X) molecule is an amino acid or a peptide, optionally chemically modified.

According to another variant said (X) molecule is a non-peptidic molecule grafted to the carboxy-terminal end of an oligopeptide.

According to another variant, an N-end module of protein destabilisation can be added to the end C-end amino-terminal of the vectorised protein.

In the simplest case where the system modules are polypeptidic in nature, it is possible to produce the whole as a recombinant protein in appropriate expression systems, prokaryotic (bacteria) or eukaryotic, for example bacculovirus). However, utilisation of a eukaryotic production system means ensuring that it does not comprise enzymatic activities resulting in UBL C-terminal cleavage. If they exist these activities will optionally have to be previously inactivated. These problems do not arise for bacteria systems, for the most part deprived of these enzymatic activities. Alternatively, it is possible to chemically synthesise the vectorised molecule by means of peptide synthesisers.

According to a variant of the invention, said vectorised molecule further comprises at least one labelling module.

This labelling module is preferably a polypeptide such as poly-histidine.

Such a labelling module could be used in particular to facilitate purification of the vectorised polypeptide, for example by affinity for metals in the case of polyhistidine.

It is noted that integration of such a labelling module into the vector is not absolutely obligatory. It can particularly be withdrawn following preparation of the vectorised molecule, before being used.

According to yet another variant of the invention, said vectorised molecule further comprises at least a cellular labelling module. It is in fact often desirable to vectorise molecules only in certain cellular types. Translocation peptides are already known for preferential incorporating by certain cellular types (Fujihara and Nadler, 1999), but the majority of translocation peptides seems efficacious in all tissues (Schwarze et al. 2000. Trends Cell Biol. 10, 290-295). However, even in this case, labelling towards certain defined cells can be specified for this vectorisation system, by adding additional modules to the vectorised molecule. For example, it has been shown that the presence of an RGD tripeptide (Arginine-Glycine-Aspartate), allows preferential addressing of metastatic cancerous cells, overexpressing integrins αν/β3.

Together the specific devices of this system (release and programmed destruction of the vector) cooperate to produce the following net result: natural peptides of any composition and sequence are directed to the very centre of the cells, clear of any attached molecules, and without alteration to the organism.

The invention will be better understood from the attached diagrams, in which:

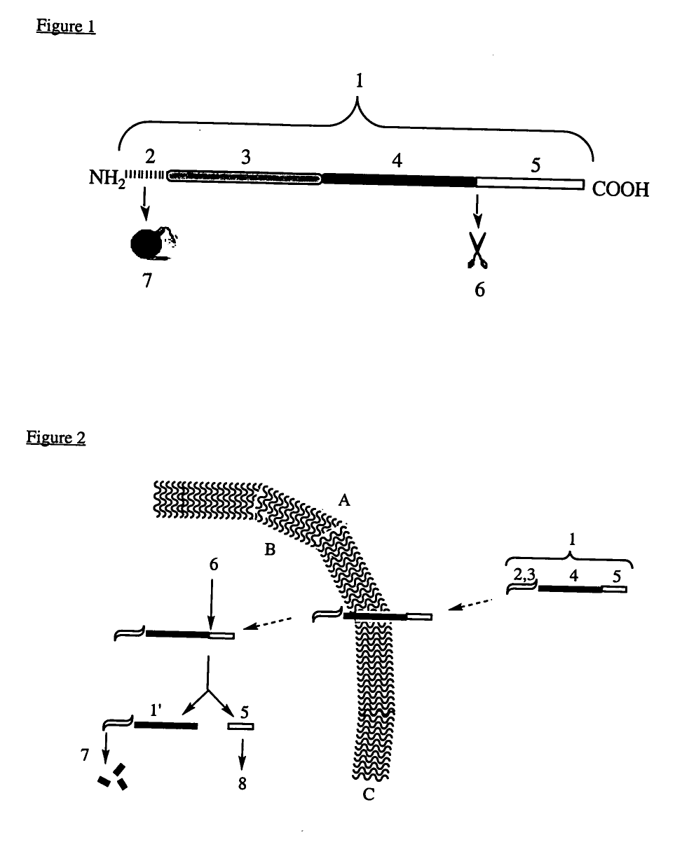

FIG. 1 illustrates a molecule vectorised according to the present invention;

FIG. 2 diagrammatically illustrates the function of the invention;

FIG. 3 diagrammatically illustrates methods A, B, C and D for exposing a particular amino acid at the N-end of the vectorised molecule (see above);

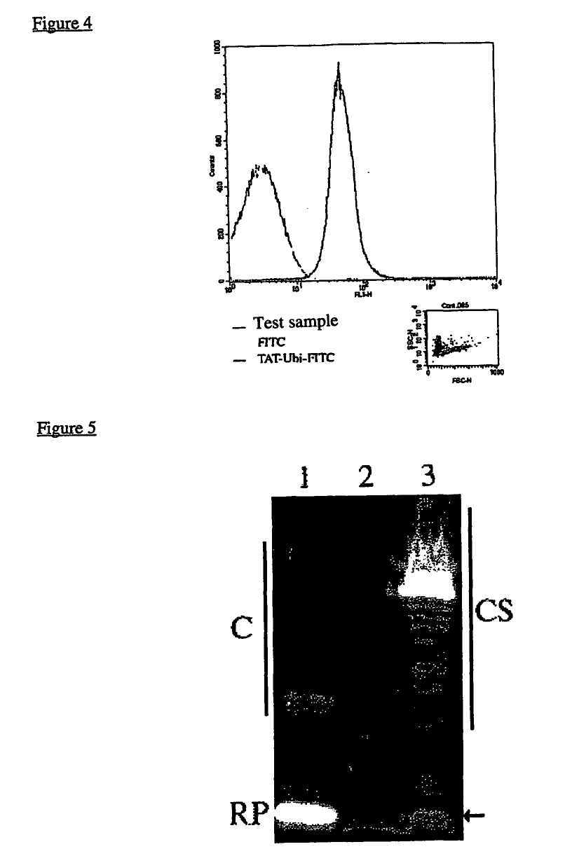

FIG. 4 illustrates a spectrum translating the incorporation of TAT-Ubi proteins coupled to the FITC, in thymocytes;

FIG. 5 illustrates a polyacrylamide gel transferred to membrane and observed on a fluorescent reader.

With reference to FIG. 1, a vectorised molecule is constituted by fusion, of NH2 to COOH, of an intracellular destabilisation module 2, a cellular incorporation module 3 capable for example of being constituted by peptide derived from the TAT protein of the AIDS virus (HIV), a ubiquitin module or other relevant peptide (UBL) 4 capable of conjugation and a molecule 5 to be transported. Once it is incorporated in the cells, the vectorised molecule is cloven between 4 and 5 by specific hydrolase 6. The molecule to be transported 5 can then exert the biological activity for which it has been designed. After liberation of the molecule to be transported 5, the rest of the vectorised molecule 2-3-4 is destroyed by an intracellular enzymatic system 7 recognising the destabilisation module 2.

With reference to FIG. 2, the vectorised molecule 1 is introduced to the extracellular A medium. Due to the cellular incorporation module 2 which it contains, this molecule crosses the cellular membrane C and reappears in the cytoplasm B. Owing to a specific hydrolase 6, the peptide bond between the molecule 5 and the UBL 4 is broken and the molecule 5 is released with its free amino-terminal end. This molecule can exert the biological activity for which it has been designed 8. The evolution of the vector 1′, constituted by fusion of modules 2, 3, 4, no longer has any influence on the function of the transported molecule 5. According to a variant, the presence of a destabilising module 2 induces destruction of the vector 1′ by intracellular machinery.

The invention has a large range of applications, from basic research to therapy. Since it imposes no condition on the nature of the X molecule, it enables polypeptides of sequences both natural and artificial to be introduced into cells. Polypeptides of natural sequence can for example correspond to domains of existing proteins. Peptides of artificial sequences can be selected such as specific ligands of known regulatory proteins, via screening strategies of random peptide banks, of direct or inverse double hybrid, or even computer-assisted modelling.

In the particular case where the peptides to be transported into the cells are precisely normal or mutated UBL, such as for example ubiquitins mutated at the level of lysine residues, the UBL module provided in the vector can be used, in which case its C-terminal extension can be any such.

The invention is also directly adaptable to screening of random peptide banks which offer billions of possible configurations. For this, a bank may be made up by directly utilising the vector described hereinabove, by inserting random nucleotide sequences at 3′ of the region coding the UBL.

The molecular tools defined in the present invention: intracellular release of the transported X molecule and programmed destruction of the other modules of the vectorised molecule, are adaptable to translocation modules already identified or in future development. They are also adaptable to the administration modalities which will be defined for the cellular incorporation modules.

In the domain of medicine, the present invention can be adapted to treating any type of pathology, from the moment when efficacious transported X molecules are able to be defined for these pathologies.

The present invention benefits from cellular activities duly confirmed in different domains of biology, such that its functionality is largely ensured.

The functionalities of the present invention are specified below.

Exposure of Any Amino Acid After Cleavage by the Xa Factor:

The possibility of using the Xa factor for exposing the amino acid wanted at the N-end of a recombinant protein is guaranteed by its cleavage specificity of this protease. Cutting the FXa is directed by recognition of sequence of four amino acids (preferably IEGR) integrally contained from NH2 of its cutting site, which does not impose any condition on the nature of the following amino acid. The Xa factor is available commercially and widely used.

Release of the Transported X Molecule Inside the Eukaryotic Cells:

This release is ensured by UBL C-terminal cleavage. The efficacy and specificity of the enzymatic activities responsible for these cleavages are fully guaranteed by their biological importance. In fact the C-terminal extensions of the UBL must absolutely be eliminated to allow ulterior conjugation of the UBL on their substrates.

Programmed Destruction of the Vector Following Cellular Incorporation:

No exception has been reported in terms of efficacy of the intracellular degradation of the proteins having a destabilising amino acid at their N-terminal end. Just like the C-terminal cleavage of the UBL, this mechanism is a natural process of any eukaryotic cell, assuring normal physiological functions. For example, this mechanism would be implied in:

degradation of secreted proteins which would be badly placed in the cellular cytoplasm, or releases of residues of protein cleavage.

large-scale protein degradation in damaged neurones. In this case intracellular ligases graft a destabilising supernumerary amino acid (often arginine) to the N-end of the proteins to be destroyed.

Stability of Molecules Containing a UBL and a DN in Extracellular Medium:

The enzymatic activities of cleavage and protein destruction, solicited respectively by the UBL and DN modules, exist only inside eukaryotic cells. It is therefore excluded that the presence of these modules causes any alteration to the vectorised molecules inasmuch as they are in the extracellular medium.

Membrane Permeability:

The properties of transmembrane transport are also duly confirmed for numerous translocation peptides according to the prior art.

Production examples of production systems of the vectors according to the present invention are detailed hereinbelow:

Several plasmids were made for production in bacteria of recombinant proteins according to the present invention.

These plasmids are only non-Iimiting examples of applying the present invention.

First Plasmid.

The plasmid described hereinbelow enables production in bacteria of a so-called ‘base’ recombinant protein, comprising all the modules of the vector, but not transported X molecules. The production of recombinant proteins for the transport of desired X proteins or peptides requires insertion into this plasmid of the nucleotide sequence coding these proteins or peptides, according to classic techniques of nucleotide cloning.

The plasmid is fabricated from the construction pQE-30 marketed by QIAGEN for bacterian protein production.

In this construction, the sequence coding ubiquitin has been imported de Xenopus laevis and not from mammals, for better use of codons in bacteria. The original kind of the coding sequence of ubiquitin can be freely selected since it does not affect the derived amino acids sequence (ubiquitin is retained at 100% between the vertebrae).

This plasmid helps produce in bacteria according to standard protocols a base protein, purified by affinity on nickel. The amino acids sequence of this protein is as follows, in letter code:

| | | |
|---|---|---|
| 1- | NRGSHHHHHHGSKLIEGRQLGYGRKKRRQR- | 30 |
| 31- | RRGGSASSHMQIFVKTLTGKTITLEVEPSD- | 60 |
| 61- | TIENVKAKIQDKEGIPPDQQRLIFAGKQLE- | 90 |
| 91- | DGRTLSDYNIQKESTLHLVLRLRGGAC- | 117 |

wherein:

The poly-histidine tag, allowing the recombinant protein to be purified selectively by affinity for metals, is the region <RGSHHHHH>. In addition to this, there are specific commercial antibodies directed against this epitope (QUIAGEN).

The recognition unit of the protease Xa factor is <IEGR>. Because the cleavage site is located on the carboxy side of the R residue, cutting by the Xa factor leads to exposing P amino acid glutamine <Q> on the end of the residual recombinant protein. The destabilising function of this amino acid can take effect in terms of the presence of K lysine residues nearby.

The translocation peptide is the TAT peptide derived from the HIV virus <YGRKKRRQRRR>.

The UBL module is ubiquitin <MQIFVKTLTG KTITLEVEPS DTIENVKAKI QDKEGIPPDQ QRLIFAGKQL EDGRTLSDYN IQKESTLHLV LRLRGG>

The transported X molecule is the dipeptide <AC>, of no biological interest and intended to be replaced by bioactive peptides or proteins, after insertion of additional sequences coding in the plasmid. The carboxy-terminal cystein residue of this dipeptide is provided in the event where it would be envisaged to graft on its thiol group a non-peptide molecule as claimed in claim 14. No other cystein exists in the base recombinant protein, so as to facilitate such a coupling reaction. It should be noted that alanine <A> is a stabilising amino acid.

In the selected example the presence of a glutamine at the amino-terminal end will fix a half-life for the recombinant protein at around 10 minutes after it is introduced to the cells, while X is released between 1 and 2 minutes after cellular incorporation.

A process for manufacturing a recombinant protein wherein the dipeptide AC is replaced by any peptide or protein is explained hereinbelow.

The sequence coding this peptide or protein is prepared in the form of double-strand ADN having at the end 5′ of the coding region with a cleavage having a flush end, and on the side 3′ a cohesive end corresponding to one of the restriction enzymes present in the plasmid (in this case Kpn I, Sal I, or Pst I). The plasmid is prepared by double cleavage on one side by the Sfo I enzyme, and on the other side with the enzyme whereof the cohesive end corresponds to that selected for the insert. The Sfo I double-strand cleavage site is localised precisely at 3 of the last codon of the ubiquitin (marked v on the following diagram).

The following diagram represents the sequences coding the COOH end of the base protein:

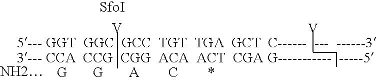

The cleavage sites by the restriction enzymes used for cloning are marked (v). In the selected example the first site (Sfo I) frees ends with flush ends, and the second site frees ends with cohesive ends. The amino acids GG correspond to the COOH limit of the ubiquitin. The dipeptide AC is the C-terminal extension of the ubiquitin in the base protein. * represents a codon “stop’ (end of translation).

The following diagram shows insertion of the sequence coding any protein in C-terminal fusion with ubiquitin.

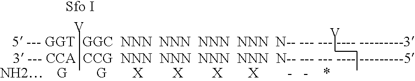

After Sfo I cleavage the inserted nucleotide sequence, illustrated with “N”, is bound to the sequence coding the ubiquitin. This bonding allows production of X proteins in C-terminal fusion of the ubiquitin, provided having managed to retain the translational reading phase between the sequence coding the ubiquitin and that of the C-terminal peptide. The insert coding the X molecule can originate from another plasmid, a reaction of PCR, or else nucleotide synthesis, in the case of peptides of reasonable size.

Second Plasmid.

A constructional variant has been made, wherein the TAT peptide of wild sequence is replaced by a version modified according to Ho and coll. (2001. Cancer Res. 61, 474-477), whereof the properties of cellular permeability are improved, and whereof the amino acid sequence is YARAAARQARA.

This plasmid codes the following protein:

| | | |
|---|---|---|
| 1- | MRGSHHHHHHGSIEGRQKYAHAAARQARAG- | 30 |
| 31- | SASSHMQIFVKTLTGKTITLEVEPSDTIEN- | 60 |
| 61- | VKAXIQDKEGIPPDQQRLIFAGKQLEDGRT- | 90 |
| 91- | LSDYNIQKESTLHLVLRLRGGAC- | 113 |

Finally a third version was constructed, wherein the methionine residue corresponding to the first amino acid of the ubiquitin is replaced by a leucine (the ATG codon was replaced by CTG). This construction is for testing the possibility of exposing a destabilising amino-terminal amino acid by removing the initial methionine by reaction to cyanogen bromide, according to a variant of the method presented in FIG. 3B.

By way of illustration of the present invention the experiments detailed hereinbelow were performed.

‘Poly-histidine-TAT-ubiquitin-extension C-terminal’ recombinant proteins were incorporated efficaciously by cells basically refractory to classic transfection techniques: normal thymocytes in primary culture, extracted from 4-week mice.

Good incorporation was visualised in two ways:

By immunological detection of the recombinant protein in cellular extracts previously incubated with recombinant proteins then washed. This detection was carried out by the western blot technique using primary antibodies directed against the peptide RGSHHHHHH (marketed by QUIAGEN).

By incorporation of fluorescence. The recombinant proteins ‘poly-histidine-TAT-ubiquitin-extension C-terminal’ were cut covalently to the fluorescein (FITC), then cleared of the free fluorescein. After incubation of mice thymocytes with these fluorescent recombinant proteins, cellular incorporation of the fluorescence was measured by fluorimetry (FACS Calibur Becton Dickinson). This measuring effectively verified that the whole of the thymocytes incorporated the protein, homogeneously and rapidly.

The intracellular liberation of the C-terminal extension of the ubiquitin was verified as follows: proteins extracted from thymocytes previously incubated with the “poly-histidine-TAT-ubiquitin-extension C-terminal’ protein marked at FITC, were subjected to electrophoresis. Examination of this electrophoresis gel in fluorescence revealed that the fluorescent ubiquitin was conjugated covalently on protein cellular substrates.

This result confirms cellular incorporation of the recombinant protein, since peptide conjugation calls on intracellular machinery.

This result confirms C-terminal cleavage of ubiquitin and thus the release of its C-terminal extension. In fact, conjugation of the ubiquitin can be performed only after exposure of its authentic C-terminal end, terminated by a dipeptide GG (GlyGly).

These experiments will be better understood with the following figures.

FIG. 4: Thymocytes extracted from young mice aged 4 weeks were incubated or not (test specimen) with FITC alone, or TAT-Ubi protein coupled to FITC, for 15 minutes. After washing the cells were analysed by flux cytometry. The spectrum illustrated in FIG. 4 shows that the protein is incorporated rapidly and homogeneously by the cells of the population.

FIG. 5: To demonstrate intracellular cleavage of the recombinant protein at the C-terminal end of the ubiquitin module thymocytes in primary culture were incubated or not with TAT-Ubi-extension C-terminal recombinant proteins cut at the FITC. Protein extracts were prepared from these cells, then subjected to denaturising polyacrylamide gel electrophoresis (SDS-PAGE, keeping only the covalent bonds). The content of the gel was transferred to membrane and observed on a fluorescence reader (scanner Storm, Molecular Dynamics). The result is illustrated in FIG. 5 in which different channels can be distinguished.

Channel 1. TAT-Ubi-extension C-terminal recombinant protein produced in bacteria and purified by affinity for nickel, then coupled to the fluorescein (FITC). The recombinant protein is clearly visible (RP). Some contaminating bacterian proteins (C) of high molecular weight are visible.

Channel 2. Proteins extracted from thymocytes test samples.

Channel 3. Proteins extracted from thymocytes incubated with preparation of recombinant protein shown in channel 1.

A band of size equivalent to RP (arrow) is clearly detected in the cells, but the majority of the fluorescence is found at the level of proteins of higher molecular weight (CS). These bands must correspond to substrates conjugation of fluorescent ubiquitin derived from the recombinant protein. The highly intense band corresponds to a non-identified protein, mainly in the thymocytes.

This result shows that:

the recombinant protein is effectively incorporated by the thymocytes

the recombinant protein is efficaciously cleaved at the C-terminal end of the ubiquitin field. In effect, conjugation of ubiquitin on intracellular protein substrates requires that the authentic end of the ubiquitin is exposed and cleared of its C-terminal extension.

### REFERENCES

Bachmair A, Finley D, Varshavsky A. 1986. In vivo half-life of a protein is a function of its amino-terminal residue. Science 234, 179-186

Fujihara, S. M., and Nader, S. G. (1999) Intranuclear targeted delivery of functional NP-kappaB by 70 kDa heat shock protein. EMBO J. 18, 411-419.

Futaki, S., Suzuki, T., Ohashi, W., Yagami, T., Tanaka, S., Ueda, K., and Sugiura, Y. (2000). Arginine-rich peptides: An abundant source of membrane-permeable peptides having potential as carriers for intracellular protein delivery. J. Biol. Chem. 276, 5836-5840.

Glotzer M, Murray A W, and Kirschner M W. (1991). Cyclin is degraded by the ubiquitin pathway. Nature 349, 132-138.

H o A, Schwarze S R, Mermelstein S J, Waksman G, Dowdy S F (2001). Synthetic protein transduction domains: enhanced transduction potential in vitro and in vivo. Cancer Res. 61,474-477.

Lund and coll. 1985. Nucleotide sequence analysis of a cDNA encoding human ubiquitin reveals that ubiquitin is synthesized as a precursor. J. Biol. Chem. 260, 7609-7613

Mayer and coll. 1989. Detection, resolution, and nomenclature of multiple ubiquitin carboxyl-terminal esterases from bovine calf thymus. Biochemistry 28, 166-172

Pickart and Rose 1985. Ubiquitine carboxyl-terminal hydrolase acts on ubiquitin carboxyl-terminal amides. J. Biol. Chem. 260, 7903-7910.

Schwarze, S. R., Hruska, K. A., and Dowdy, S. F. (2000). Protein transduction: unrestricted delivery into all cells? Trends Cell Biol. 10, 290-295

1. A vector for introducing a (X) molecule having at least one amino-terminal end into a eukaryotic cell, characterised in that it is constituted by association of a cellular incorporation module enabling membrane translocation, and a UBL module, that is, a ubiquitin module or other related peptide capable of conjugation or a version of modified or shortened UBL, said vector having a carboxy-terminal end for fusing by peptide bond to the amino-terminal end of said (X) molecule

2. The vector as claimed in claim 1, characterised in that it contains an intracellular destabilisation module.

3. The vector as claimed in claim 2, characterised in that said intracellular destabilisation module is constituted by a “destruction box” or by an amino-terminal end exposing destabilising amino acid followed by a region comprising one or more lysines.

4. The vector as claimed in claim 3, characterised in that said amino acid amino-end is glutamine.

5. The vector as claimed in claim 3, characterised in that a destabilising molecule (X) amino acid is exposed to the N-end end after cleavage by a protease.

6. The vector as claimed in claim 5, characterised in that said protease is the FXa factor.

7. The vector as claimed in any one of claims 1 to 6, characterised in that it contains a labelling module.

8. The vector as claimed in claim 7, characterised in that said labelling module is a polypeptide.

9. The vector as claimed in claim 8, characterised in that said labelling module is poly-histidine.

10. The vector as claimed in any one of claims 1 to 9, characterised in that said incorporation module is a membrane translocation peptide.

11. The vector as claimed in claim 10, characterised in that said incorporation module is a peptide derived from the TAT protein of the AIDS virus (HIV).

12. The vector as claimed in any one of claims 1 to 11, characterised in that the UBL module is a ubiquitin module.

13. A vectorised molecule, characterised in that it is constituted by fusion of a (X) molecule having at least one amino-terminal end whereof said amino-terminal end is fused by a peptide bond to the carboxy-terminal end of the vector as claimed in any one of claims 1 to 12.

14. The vectorised molecule as claimed in claim 13, characterised in that said (X) molecule is an amino acid or a peptide.

15. The vectorised molecule as claimed in claim 14, characterised in that said (X) molecule is an amino acid or a chemically modified peptide.

16. The vectorised molecule as claimed in claim 15, characterised in that said (X) molecule is a non-peptidic molecule grafted to the carboxy-terminal end of an oligopeptide.
